SEQUENCE LISTING (6 sequences)

SEQ ID NO: 1 - DNA, 387 bp, unidentified
  source 1..387: mol_type = genomic DNA
ggtggttcaagatctgtcgacgaagtcaaggtaacagacacaccgaaaagatccaggagagattttggactcgactgtgatgagcactcaacagaatctcgatgctgtcgttaccctctaactgtggattttgaagcttttggatgggactggattattgcacccaaaagatataaggccaattactgctctggagagtgtgaatttgtatttttacaaaaataccctcacactcatcttgtgcaccaagcaaaccccagaggttcagcaggcccctgctgtactcccacaaagatgtctccaatcaatatgctatattttaatggcaaagaacaaataatatatgggaaaattccagccatggtagtagatcgctgtgggtgctca

SEQ ID NO: 2 - DNA, 15 bp, unidentified
  source 1..15: mol_type = genomic DNA
ccagccccagctcca

SEQ ID NO: 3 - DNA, 714 bp, unidentified
  source 1..714: mol_type = genomic DNA
ggtttggaatgtgatggtaaagttaatatttgttgtaaaaaacaattttttgttagttttaaagatattggttggaatgattggattattgctccaagtggttatcatgctaattattgtgaaggtgaatgtccaagtcatattgctggtaccagtggtagtagtttgagttttcatagtaccgttattaatcattatcgtatgcgtggtcatagtccatttgctaatttgaaaagttgttgtgttccaaccaaattgcgtccaatgagtatgttgtattatgatgatggtcaaaatattattaaaaaagatattcaaaatatgattgttgaagaatgtggttgtagtggtggttcaagatccggtttggaatgtgatggtaaagttaatatttgttgtaaaaaacaattttttgttagttttaaagatattggttggaatgattggattattgctccaagtggttatcatgctaattattgtgaaggtgaatgtccaagtcatattgctggtaccagtggtagtagtttgagttttcatagtaccgttattaatcattatcgtatgcgtggtcatagtccatttgctaatttgaaaagttgttgtgttccaaccaaattgcgtccaatgagtatgttgtattatgatgatggtcaaaatattattaaaaaagatattcaaaatatgattgttgaagaatgtggttgtagttaa

SEQ ID NO: 4 - DNA, 348 bp, unidentified
  source 1..348: mol_type = genomic DNA
ggtttggaatgtgatggtaaagttaatatttgttgtaaaaaacaattttttgttagttttaaagatattggttggaatgattggattattgctccaagtggttatcatgctaattattgtgaaggtgaatgtccaagtcatattgctggtaccagtggtagtagtttgagttttcatagtaccgttattaatcattatcgtatgcgtggtcatagtccatttgctaatttgaaaagttgttgtgttccaaccaaattgcgtccaatgagtatgttgtattatgatgatggtcaaaatattattaaaaaagatattcaaaatatgattgttgaagaatgtggttgtagt

SEQ ID NO: 5 - DNA, 15 bp, unidentified
  source 1..15: mol_type = genomic DNA
ccagccccagctcca

SEQ ID NO: 6 - DNA, 432 bp, unidentified
  source 1..432: mol_type = genomic DNA
gtttttcttcaaaagtatccacatacacatcttgttcatcaagctggtggttcaagatctgtcgacgaagtcaaggtaacagacacaccgaaaagatccaggagagattttggactcgactgtgatgagcactcaacagaatctcgatgctgtcgttaccctctaactgtggattttgaagcttttggatgggactggattattgcacccaaaagatataaggccaattactgctctggagagtgtgaatttgtatttttacaaaaataccctcacactcatcttgtgcaccaagcaaaccccagaggttcagcaggcccctgctgtactcccacaaagatgtctccaatcaatatgctatattttaatggcaaagaacaaataatatatgggaaaattccagccatggtagtagatcgctgtgggtgctca

(19) United States
(12) Patent Application Publication
(10) Pub. No.: US 2025/0312395 A1
(43) Pub. Date: Oct. 9, 2025

(54) PHARMACEUTICAL COMPOSITION FOR ALLEVIATIONN, TREATMENT, AND PREVENTION OF SARCOPENIA CONTAINING MICROORGANISM TRANSFORMED WITH CELL SURFACE DISPLAY VECTOR OPERABLY LINKED WITH GENE ENCODING MYOSTATIN AND ACTIVIN A PROTEINS AS ACTIVE INGREDIENT

(71) Applicant: MOALIFEPLUS CO., LTD., Yongin-si (KR)

(72) Inventors: Hyun Jun KANG, Goyang-si (KR); Jae Chul CHOI, Daejeon (KR); Chan Hee YOO, Hwaseong-si (KR); Jae Pyeong JANG, Yongin-si (KR); Kyung Soo HAHM, Seoul (KR)

(21) Appl. No.: 18/627,462

(22) Filed: Apr. 5, 2024

(51) Int. Cl. A61K 35/747; A61K 38/18; A61P 21/00

(52) U.S. Cl. CPC A61K 35/747; A61K 38/18; A61P 21/00

(57) ABSTRACT

The present invention relates a pharmaceutical composition for alleviation, treatment, and prevention of sarcopenia, including, as an active ingredient, a microorganism transformed with a cell surface display vector operably linked with a gene encoding myostatin and activin A proteins.

### BACKGROUND OF THE INVENTION

### Field of the Invention

The present invention relates to a cell surface display vector for alleviation, treatment, and prevention of sarcopenia, the vector expressing myostatin and activin A as a bispecific antigen, and a microorganism transformed with the same.

### Description of the Related Art

Myostatin, which is a growth regulator that selectively down-regulates skeletal muscle growth (negative regulator), belongs to the TGF-β superfamily and consists of 375 amino acid precursors. In vertebrates, a method of producing an antibody against a myostatin immunogen may reduce endogenous myostatin activity in vertebrates, thereby exhibiting biological effects such as increased body weight, increased muscle mass, increased number of muscle cells, increased muscle cell size, decreased body fat mass, and increased muscle strength.

In addition, activin A, which is a member of the TGF-β superfamily, is known to have a wide range of biological activities such as mesoderm induction, neuronal differentiation, bone remodeling, and hematopoiesis.

Meanwhile, cell surface display technology is a technology for displaying a foreign protein on the surface using a surface protein of microorganisms such as bacteria or yeast as a surface anchoring motif, and it has significant industrial application potential. In particular, there has never been an invention to display myostatin and activin A as a bispecific antigen on a cell surface and use them as an active substance for the treatment of sarcopenia.

### SUMMARY OF THE INVENTION

The present invention is to provide a pharmaceutical composition including, as active ingredients, a cell surface display vector for alleviation, mitigation, treatment, and prevention of sarcopenia, the vector expressing myostatin and activin A as a bispecific antigen, and a microorganism transformed with the same.

To accomplish the above purpose, the present invention may provide a pharmaceutical composition for alleviation, treatment, and prevention of sarcopenia, containing, as an active ingredient, a microorganism transformed with a cell surface display vector operably linked with a gene encoding myostatin and activin A proteins.

In addition, in the present invention, the cell surface display vector may include a pgsA gene.

In addition, in the present invention, the myostatin may have a gene sequence of SEQ ID NO: 1.

In addition, in the present invention, the activin A may have a gene sequence of SEQ ID NO: 3.

In addition, in the present invention, the microorganism may be a lactic acid bacterium, and the lactic acid bacterium may be Lactobacillus paracasei.

### BRIEF DESCRIPTION OF THE DRAWINGS

FIG. 1 shows a pKV-Pald-pgsA-Myostatin-Linker-ActivinA (pKV-pgsA-Myo-Act) vector gene sequence.

FIG. 2 shows a pKV-Pald-pgsA-ActivinA-Linker-Myostatin (pKV-pgsA-Act-Myo) vector gene sequence.

FIG. 3 shows a Western blot experiment to confirm the expression of a bispecific antigen in a transformed lactic acid bacterium.

FIGS. 4 and 5 show a stabilization step of a transformed lactic acid bacterium.

FIGS. 6 and 7 show the expression levels of myostatin and activin A in a membrane fraction.

FIG. 8 shows an experimental schedule for evaluating the efficacy in a sarcopenia animal model.

FIGS. 9 and 10 show a comparison of the results of a grip strength test and a rotarod latency test between the lactic acid bacterium G1 (L. paracasei_Mcs), which does not express an antigen, and the experimental group G2 (L. paracasei_Myo-Act, 10 mg).

FIGS. 11 and 12 show a comparison of the results of a histopathological test between the lactic acid bacterium G1 (L. paracasei_Mcs), which does not express an antigen, and the experimental group G2 (L. paracasei_Myo-Act, 10 mg).

FIG. 13 shows an experimental schedule for evaluating the efficacy in an aged mouse model.

FIGS. 14 and 15 show a comparison of the results of a grip strength test and a rotarod latency test between the lactic acid bacterium G2 (L. paracasei_MCS), which does not express an antigen, and the experimental group G3 (L. paracasei_Myo-Act, 10 mg).

FIG. 16 shows a comparison of the results of a histopathological test between the lactic acid bacterium G2 (L. paracasei_MCS), which does not express an antigen, and the experimental group G3 (L. paracasei_Myo-Act, 10 mg).

### DETAILED DESCRIPTION OF THE EMBODIMENTS

As one embodiment of the present invention, provided is a pharmaceutical composition for alleviation, treatment, and prevention of sarcopenia, containing, as an active ingredient, a microorganism transformed with a cell surface display vector operably linked with a gene encoding myostatin and activin A proteins.

The composition may include a pharmaceutically acceptable carrier in addition to the active ingredient, and the carrier is one that is commonly used in the preparation of pharmaceutically acceptable ingredients and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil, but is not limited thereto. In addition to the above ingredients, the pharmaceutical composition of the present invention may further include lubricants, wetting agents, sweeteners, flavoring agents, emulsifiers, suspending agents, preservatives, and the like. Suitable pharmaceutically acceptable carriers and formulations are described in detail in Remington's Pharmaceutical Sciences (19th ed., 1995).

In addition, an appropriate dosage of the pharmaceutical composition according to the present invention may be prescribed diversely depending on factors such as the formulation method, administration method, a patient's age, weight, sex, pathological conditions, diet, administration time, route of administration, excretion rate, and response sensitivity.

In addition, the pharmaceutical composition of the present invention may be administered orally or parenterally, and when administered parenterally, it may be administered by intravenous injection, subcutaneous injection, intramuscular injection, intraperitoneal injection, transdermal administration or the like, and it is preferable that the route of administration is determined depending on the type of disease to which the pharmaceutical composition of the present invention is applied.

In addition, the pharmaceutical composition according to the present invention is formulated using a pharmaceutically acceptable carrier and/or excipient according to a method that may be easily performed by a person skilled in the art to which the present invention pertains, so it may be prepared in the form of a unit dose or prepared by placing it into a multi-dose container. At this time, the dosage form may be in the form of a solution, suspension, or emulsion in an oil or aqueous medium, or may be in the form of an extract, powder, granule, tablet, or capsule, and may further include a dispersant or stabilizer.

In addition, the pharmaceutical composition according to the present invention may further include carriers and vehicles commonly used in the pharmaceutical field. Specifically, it may include ion exchange resins, alumina, aluminum stearate, lecithin, serum proteins (e.g., human serum albumin), buffering substances (e.g., various phosphates, glycine, sorbic acid, potassium sorbate, partial glyceride mixtures of saturated vegetable fatty acids), water, salts or electrolytes (e.g. protamine sulfate, disodium hydrogen phosphate, calcium hydrogen phosphate, sodium chloride, and zinc salts), colloidal silica, magnesium trisilicate, polyvinylpyrrolidone, cellulosic matrices, polyethylene glycol, sodium carboxymethylcellulose, polyarylate, wax, or lanolin, but is not limited thereto.

In addition, the pharmaceutical composition according to the present invention may be in the dosage forms of granules, powder, coated tablets, tablets, capsules, suppositories, syrups, juices, suspensions, emulsions, drops, injections, or sustained-release dosage forms of an active compound, and it may be administered in various oral or parenteral dosage forms. When formulated, it may be prepared using fillers, extenders, binders, wetting agents, disintegrants, diluents such as surfactants or excipients commonly used in the pharmaceutical field.

In one embodiment of the present invention, the present invention may provide a pharmaceutical composition for alleviation, treatment, and prevention of sarcopenia, containing, as an active ingredient, a microorganism transformed with a cell surface display vector operably linked with a gene encoding myostatin and activin A proteins.

In another embodiment of the present invention, the cell surface display vector may include a pgsA gene.

In still another embodiment of the present invention, in the present invention, the myostatin may have a gene sequence of SEQ ID NO: 1.

In yet another embodiment of the present invention, the activin A may have a gene sequence of SEQ ID NO: 3.

In yet another embodiment of the present invention, the microorganism may be a lactic acid bacterium, and the lactic acid bacterium may be Lactobacillus paracasei.

Hereinafter, the present invention will be described in more detail through specific examples. These examples are only for illustrating the present invention, and it will be obvious to those skilled in the art that the scope of the present invention should not be construed as limited by these examples.

### Example 1. Construction of Vector for Bispecific Antigen Expression

### Example 1-1. Construction of pKV-Pald-pgsA-Myostatin-Linker-ActivinA (pKV-pgsA-Myo-Act) Vector

A vector that co-expresses myostatin and activin A was constructed. First, a pKV-pgsA-Act vector, to which an activin A gene had already been inserted to express, was digested using restriction enzyme BamHI, and the Prodomain-mMyostatin gene amplification was performed using the primers shown in Table 1 below.

TABLE 1

| Primer | Length | Sequence |
|---|---|---|
| Pro_Myo_F | 39 mer | TGGTTCTGGTGGATCCGGTGGTTCAAGATCTGTCGACGA |
| Pro_Myo_R#1 | 52 mer | ATTCCAAACCGGATCCTGGAGCTGGGGCTGGTGAgcacccacagcgatctac |

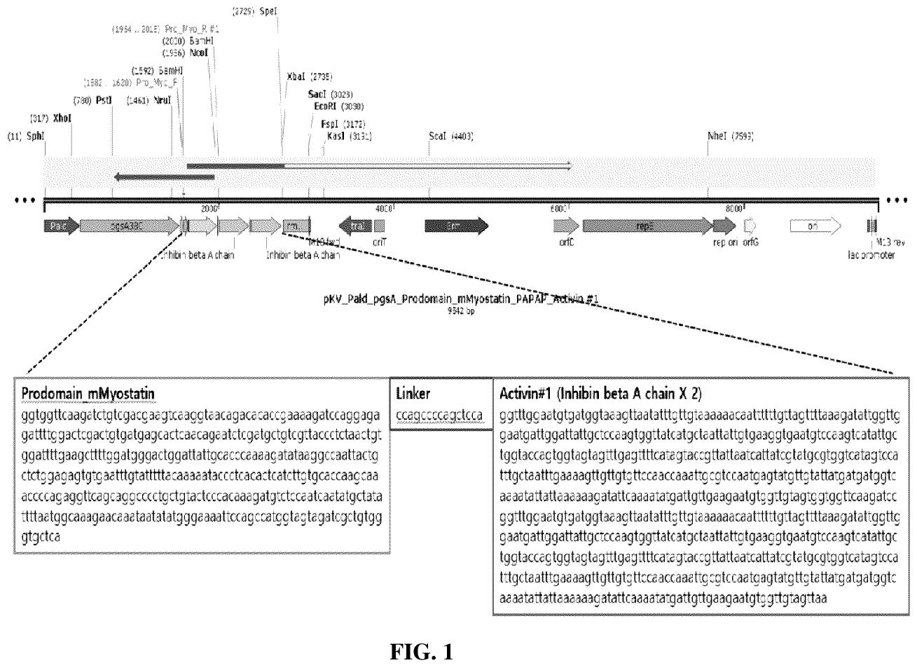

Afterwards, a pKV-Pald-pgsA-Myostatin-Linker-Activin A (pKV-pgsA-Myo-Act) vector was constructed through ligation using the In-Fusion HD kit (Clontech, USA) (FIG. 1), and the gene sequences of myostatin, the linker, and activin A are as follows.

nucleotide sequence 1-Prodomain mMyostatin
5′-GGTGGTTCAAGATCTGTCGACGAAGTCAAGGTAACAGACACACCGAAAAGATCCAG
GAGAGATTTTGGACTCGACTGTGATGAGCACTCAACAGAATCTCGATGCTGTCGTTA
CCCTCTAACTGTGGATTTTGAAGCTTTTGGATGGGACTGGATTATTGCACCCAAAAG
ATATAAGGCCAATTACTGCTCTGGAGAGTGTGAATTTGTATTTTTACAAAAATACCC
TCACACTCATCTTGTGCACCAAGCAAACCCCAGAGGTTCAGCAGGCCCCTGCTGTAC
TCCCACAAAGATGTCTCCAATCAATATGCTATATTTTAATGGCAAAGAACAAATAAT
ATATGGGAAAATTCCAGCCATGGTAGTAGATCGCTGTGGGTGCTCA-3′
nucleotide sequence 2-Linker
5′-CCAGCCCCAGCTCCA-3′
nucleotide sequence 3-Activin#1 (Inhibin beta A chain X 2)
5′-GGTTTGGAATGTGATGGTAAAGTTAATATTTGTTGTAAAAAACAATTTTTTGTTAGTT
TTAAAGATATTGGTTGGAATGATTGGATTATTGCTCCAAGTGGTTATCATGCTAATT
ATTGTGAAGGTGAATGTCCAAGTCATATTGCTGGTACCAGTGGTAGTAGTTTGAGTT
TTCATAGTACCGTTATTAATCATTATCGTATGCGTGGTCATAGTCCATTTGCTAATTT
GAAAAGTTGTTGTGTTCCAACCAAATTGCGTCCAATGAGTATGTTGTATTATGATGA
TGGTCAAAATATTATTAAAAAAGATATTCAAAATATGATTGTTGAAGAATGTGGTTG
TAGTGGTGGTTCAAGATCCGGTTTGGAATGTGATGGTAAAGTTAATATTTGTTGTAA
AAAACAATTTTTTGTTAGTTTTAAAGATATTGGTTGGAATGATTGGATTATTGCTCCA
AGTGGTTATCATGCTAATTATTGTGAAGGTGAATGTCCAAGTCATATTGCTGGTACC
AGTGGTAGTAGTTTGAGTTTTCATAGTACCGTTATTAATCATTATCGTATGCGTGGTC
ATAGTCCATTTGCTAATTTGAAAAGTTGTTGTGTTCCAACCAAATTGCGTCCAATGA
GTATGTTGTATTATGATGATGGTCAAAATATTATTAAAAAAGATATTCAAAATATGA
TTGTTGAAGAATGTGGTTGTAGTTAA-3′

### Example 1-2. Construction of pKV-Pald-pgsA-ActivinA-Linker-Myostatin (pKV-1gsA-Act-Myo) Vector

The pKV-pgsA-MCS vector was digested using restriction enzymes BamHI and XbaI, and ActivinA-Linker-Myostatin gene amplification was performed using the primers shown in Table 2 below.

TABLE 2

| Primer | Length | Sequence |
|---|---|---|
| 1x_act_F | 33 mer | TGGTTCTGGTGGATCAGGATCCGCTGAAGCTGC |
| 1x_act_R | 39 mer | AAAACACTTATCTAGTCTAGATTATGAGCACCCACAGCG |

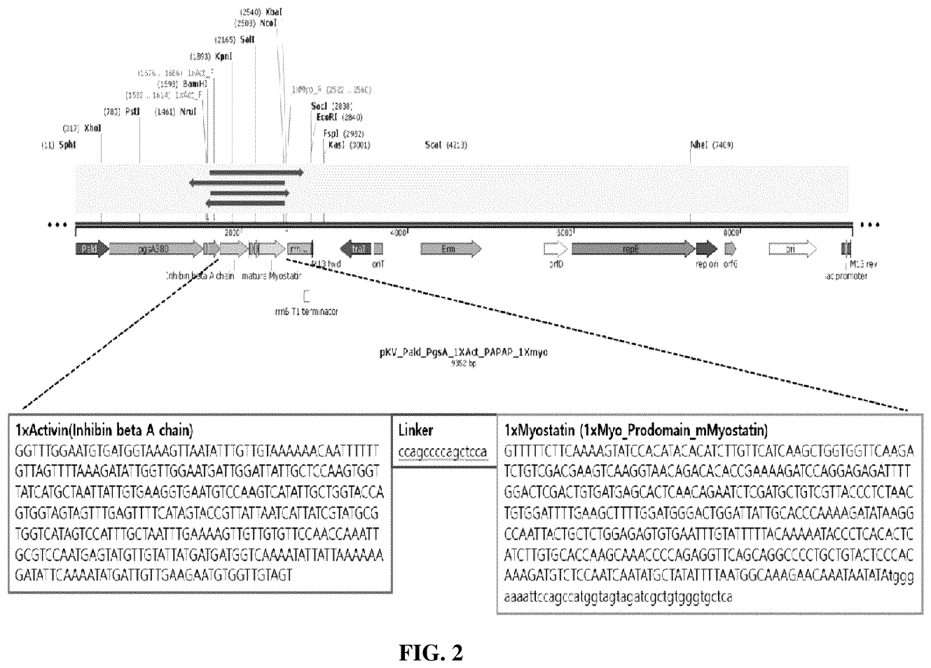

Afterwards, a pKV-Pald-pgsA-ActivinA-Linker-Myostatin (pKV-pgsA-Act-Myo) vector was constructed through ligation (FIG. 2), and the gene sequences of activin A, the linker, and myostatin are as follows.

nucleotide sequence 4-1xActivin (Inhibin beta A chain)
5′-GGTTTGGAATGTGATGGTAAAGTTAATATTTGTTGTAAAAAACAATTTTTTGTTAGTT
TTAAAGATATTGGTTGGAATGATTGGATTATTGCTCCAAGTGGTTATCATGCTAATT
ATTGTGAAGGTGAATGTCCAAGTCATATTGCTGGTACCAGTGGTAGTAGTTTGAGTT
TTCATAGTACCGTTATTAATCATTATCGTATGCGTGGTCATAGTCCATTTGCTAATTT
GAAAAGTTGTTGTGTTCCAACCAAATTGCGTCCAATGAGTATGTTGTATTATGATGA
TGGTCAAAATATTATTAAAAAAGATATTCAAAATATGATTGTTGAAGAATGTGGTTG
TAGT-3′
nucleotide sequence 5-Linker
5′-CCAGCCCCAGCTCCA-3′
nucleotide sequence 6-1xMyostatin (1xMyo Prodomain mMyostatin)
5′-GTTTTTCTTCAAAAGTATCCACATACACATCTTGTTCATCAAGCTGGTGGTTCAAGAT
CTGTCGACGAAGTCAAGGTAACAGACACACCGAAAAGATCCAGGAGAGATTTTGGA
CTCGACTGTGATGAGCACTCAACAGAATCTCGATGCTGTCGTTACCCTCTAACTGTG
GATTTTGAAGCTTTTGGATGGGACTGGATTATTGCACCCAAAAGATATAAGGCCAAT
TACTGCTCTGGAGAGTGTGAATTTGTATTTTTACAAAAATACCCTCACACTCATCTTG
TGCACCAAGCAAACCCCAGAGGTTCAGCAGGCCCCTGCTGTACTCCCACAAAGATG
TCTCCAATCAATATGCTATATTTTAATGGCAAAGAACAAATAATATATGGGAAAATT
CCAGCCATGGTAGTAGATCGCTGTGGGTGCTCA-3′

### Example 1-3. Construction of pKV-Pald-pgsA-Myostatin-Linker-INHBA-Activin A FL (pKV-pgsA-Myo-INHBA-ActA FL) Vector

A pKV-pgsA-MCS vector was digested using restriction enzyme BamHI, Myostatin-Linker-INHBA-ActivinA FL gene amplification was performed using the primers shown in Table 3 below, and then a pKV-Pald-pgsA-Myostatin-Linker-INHBA-ActivinA FL (pKV-pgsA-Myo-INHBA-ActA FL) vector was constructed through ligation.

TABLE 3

| Primer | Length | Sequence |
|---|---|---|
| mMyo_Act_F | 41 mer | TGGTTCTGGTGGATCCGATTTTGGACTCGACTGTGATGAGC |
| 4xMyo_Act_R1 | 38 mer | TACCTCATTAGGATCCCTATGAGCACCCACACTCCTCC |

### Example 2. Securing Lactic Acid Bacterium Co-Expressing Bispecific Antigen

### Example 2-1. Production of Transformed Lactic Acid Bacterium and Confirmation of Bispecific Antigen Expression

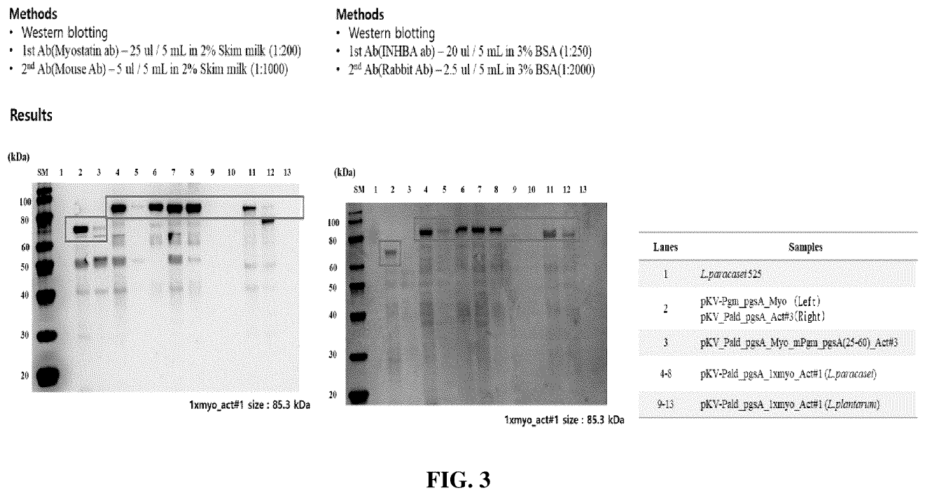

Bispecific antigen expression of the transformed lactic acid bacterium was confirmed through Western blot. Specifically, a cloned plasmid was transformed into L. paracasei 525 competent cells, which were spread on a deMan, Rogosa, and Sharpe (MRS) (erythromycin, 16 μg/ml) agar plate and cultured at 30° C. for three days. Afterwards, the cultured colony was cultured into an MRS (erythromycin, 16 μg/ml) broth and cultured at 30° C. for three days. Then, 1.4 ml of the culture solution was centrifuged to remove the supernatant, and the cells were washed three times using 1 ml of phosphate-buffered saline (PBS). Next, after resuspending the cell pellet with 250 μl of PBS, sonication was performed for 15 minutes using a wave sonicator. Then, the total protein amount was quantified using the bicinchoninic acid (BCA) protein assay to prepare a sample of 60 μg/40 μl, and 30 μg of it was loaded onto the SDS-PAGE gel. After loading 30 μg of the sample and performing electrophoresis, the cells were transferred to a polyvinylidene difluoride (PVDF) membrane to confirm the expression of myostatin and activin A, as shown in FIG. 3. According to FIG. 3, it can be confirmed that high expression occurred at the #4, #6, #7, and #8 colonies by the myostatin and activin A antibodies.

### Example 2-2. Stabilization of Transformed Lactic Acid Bacterium

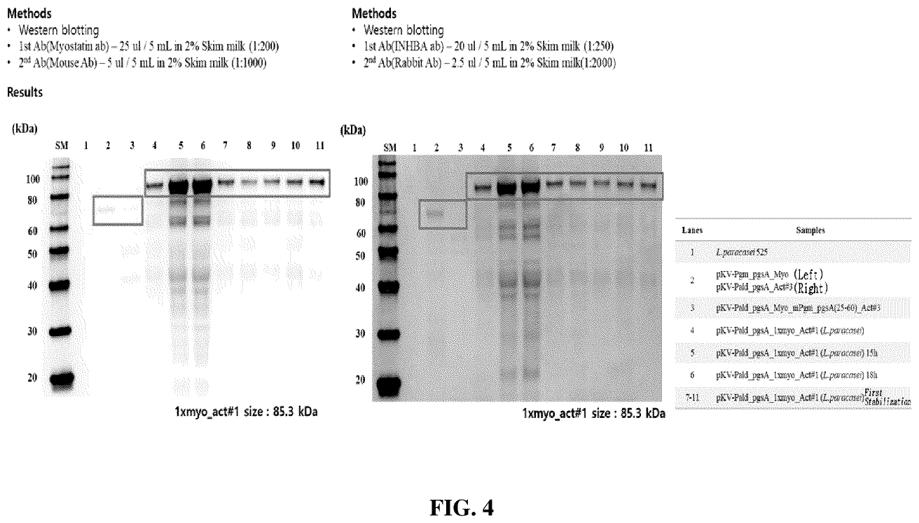

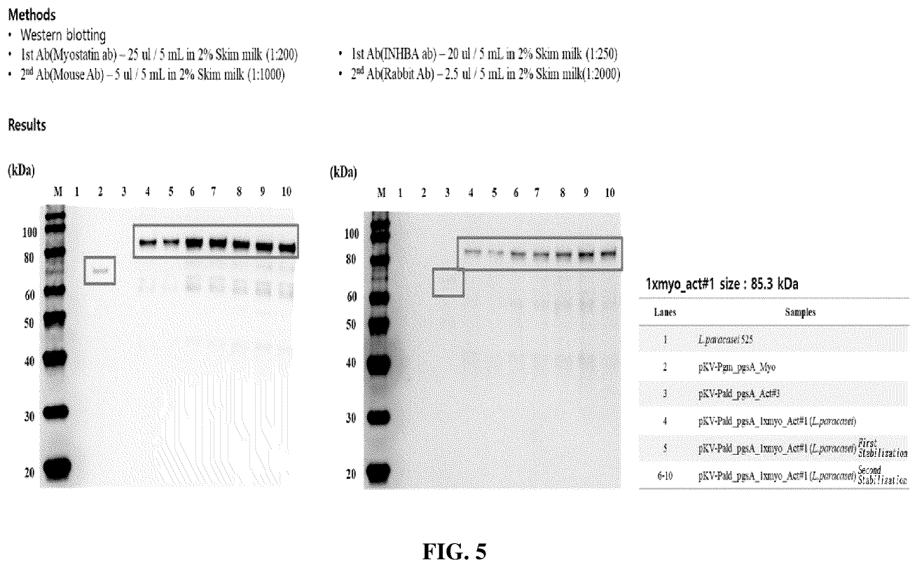

A colony culture medium was streaked onto an MRS (erythromycin, 16 ug/ml) agar plate and cultured at 30° C. for three days. Afterwards, the cultured colony was inoculated into an MRS (erythromycin, 16 μg/ml) broth and cultured at 30° C. for three days. Then, 1.4 ml of the culture was centrifuged to remove the supernatant, and the cells were washed three times using 1 ml of PBS. Next, after resuspending the cell pellet with 250 μl of PBS, sonication was performed for 15 minutes using a wave sonicator. Then, the total protein amount was quantified using the BCA protein assay to prepare a sample of 60 μg/40 μl, and 30 μg of it was loaded onto the SDS-PAGE gel. After loading 30 μg of the sample and performing electrophoresis, the cells were transferred to a PVDF membrane to confirm the expression of myostatin and activin A, as shown in FIG. 4. The results of performing second stabilization are shown in FIG. 5, from which it can be confirmed that the pKV-pgsA-Myo-Act expression increased. After the second stabilization, stocks of the lane 6 strain and the lane 7 strain were prepared and selected as Candidate Substance 1 (pKV-Pald-pgsA-Myo-Act, BLS-M32).

### Example 2-3. Confirmation of Bispecific Antigen Co-Expression in Membrane Fraction of Transformed Lactic Acid Bacterium

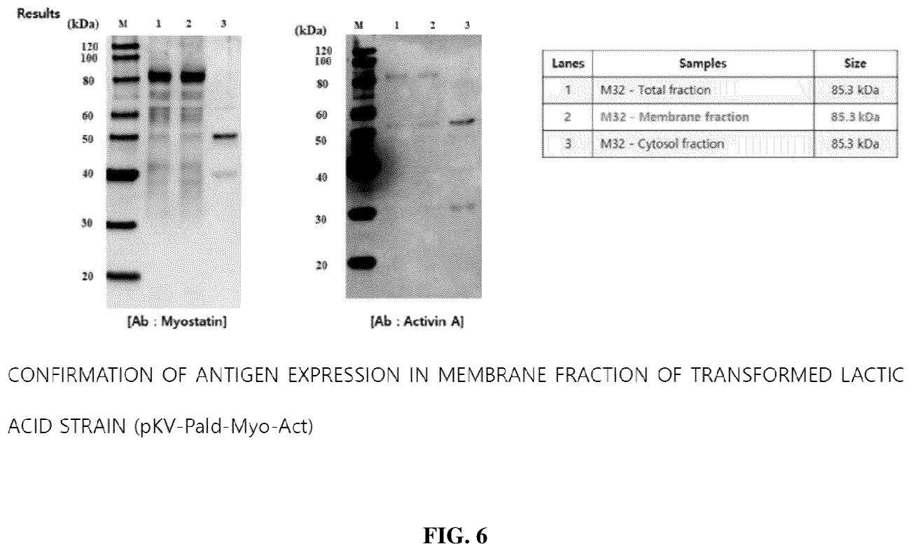

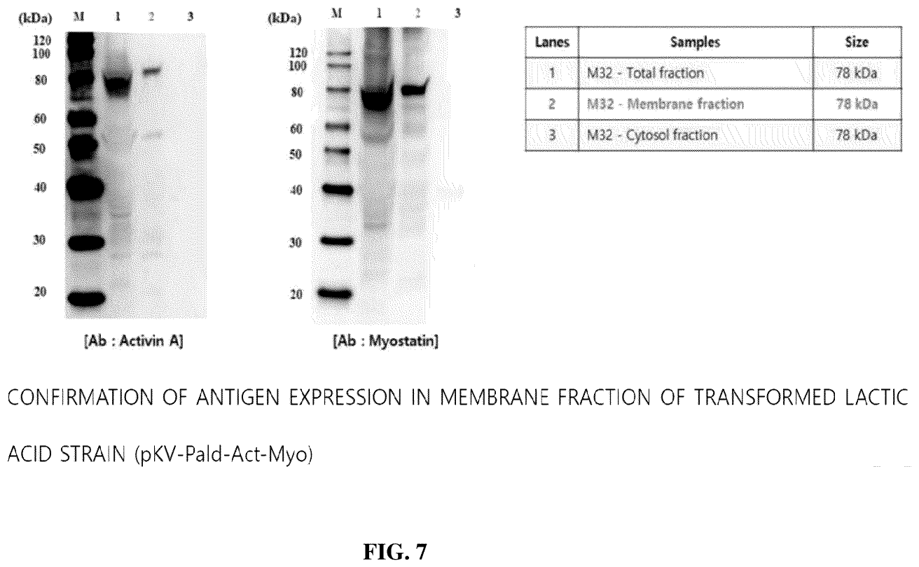

The transformed and freeze-dried lactic acid bacterium (BLS-M32) was resuspended in PBS and then sonicated for 15 minutes using a wave sonicator, and a membrane fraction and a cytosol fraction were separated using ultra-high-speed centrifugation (25,000 g, 2 h). Afterwards, the total protein amount was quantified using the BCA protein assay to prepare a sample at a concentration of 60 μg/40 μl, and 30 μg of it was loaded onto the SDS-PAGE gel. After loading 30 μg of the sample and performing electrophoresis, the cells were transferred to a PVDF membrane to confirm the expression of myostatin and activin A, as shown in FIGS. 6 and 7. It can be confirmed that myostatin and activin A were successfully expressed in the membrane fraction of the transformed lactic acid bacterium (pKV-Pald-Myo-Act and pKV-Pald-Act-Myo).

### Example 3. Confirmation of Sarcopenia Treatment Efficacy

### Example 3-1. Evaluation of Efficacy in Sarcopenia Animal Model

### Example 3-1-1. Experiment Conditions

A non-clinical efficacy test was performed using a mouse model (C57BL/10J-mdx), which is well known for exhibiting the characteristics of sarcopenia. Specifically, a killed Lactobacillus paracasei lactic acid bacterium was administered at a concentration of 10 mg/head, and after 12 weeks from the start of the administration, sacrifice was performed to confirm the outcome variables. Serum myostatin/activin A IgG antibody was measured using enzyme-linked immunosorbent assay (ELISA), and calf muscles were extracted to perform a morphological analysis of the muscle tissue. The composition of the mice used in the test is shown in Table 4 below.

TABLE 4

| | | | | | amount of | volume of |
|---|---|---|---|---|---|---|
| | | Number of | | Route of | administration | administered solution |
| Group | Sex | animals | Test substance | administration | (mg/head) | (mL/head) |
| G1 | M | 8 | L. paracasei | oral | 10 | 0.2 |
| | | | (MCS) | administration | | |
| G2 | M | 10 | L. paracasei | oral | 10 | 0.2 |
| | | | (pAld-Myo-Act) | administration | | |

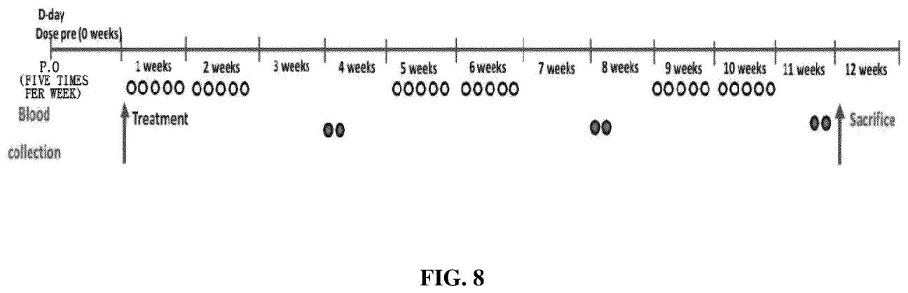

The test substance was administered a total of 30 times once per day in Weeks 1, 2, 5, 6, 9, and 10 from the start of the administration (FIG. 8). The experiment was performed by directly administering it into the stomach using an oral zonde in an amount of 200 μl/head.

### Example 3-1-2. Grip Strength Test and Rotarod Latency Test

To confirm the improvement of grip strength and muscle endurance by the composition according to the present invention, a grip strength test and a rotarod latency test were performed.

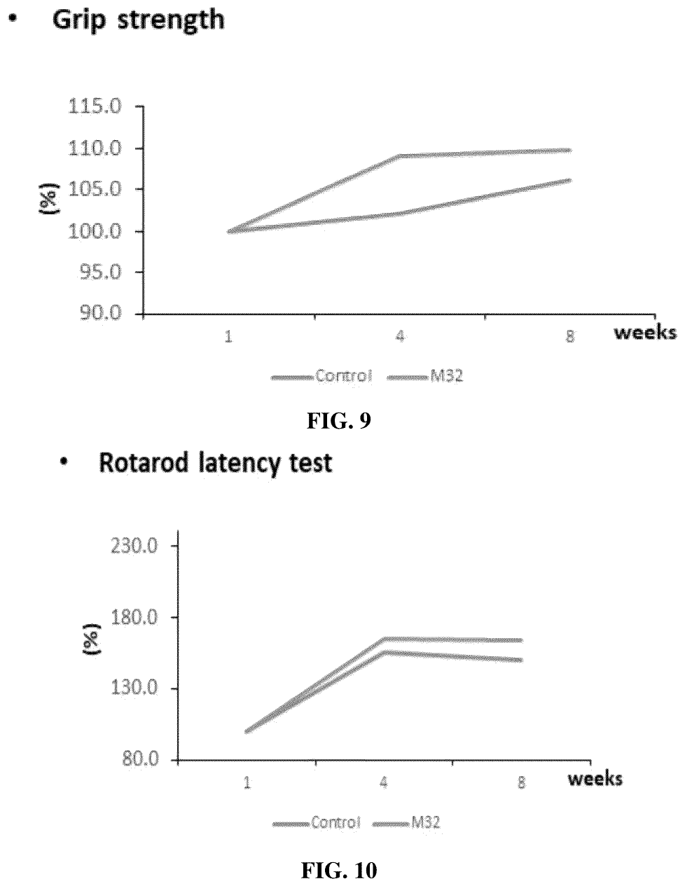

First, in the grip strength test to measure grip strength, the mouse was placed on a grip strength meter (47200, Ugo Basile, Italy) before and 4, 8, and 12 weeks after administering the test substance, and the tail was pulled to measure the grip strength. The results are shown in FIG. 9. It can be confirmed that the foreleg muscle strength (grip strength) was increased in the G2 (L. paracasei_Myo-Act, 10 mg) experimental group compared to the lactic acid bacterium G1 (L. paracasei_Mcs) control group, which did not express an antigen from four weeks after the administration of the test substance until the end of the administration.

Next, in the rotarod latency test to measure muscle endurance, the mice were placed on a rotarod-treadmill (JD-A-07MA5, B.S Technolab Inc., Korea) in 4, 8, and 12 weeks after the administration of the test substance, and the measurement was performed for 300 sec while increasing the rotational speed at regular intervals from 4 rpm to 40 rpm. The results are shown in FIG. 10, and it can be confirmed that the muscular endurance increased in the G2 (L. paracasei_Myo-Act, 10 mg) experimental group compared to the lactic acid bacterium G1 (L. paracasei_Mcs) control group, which did not express an antigen from four weeks after the administration of the test substance until the end of the administration.

### Example 3-1-3. Histopathological Examination

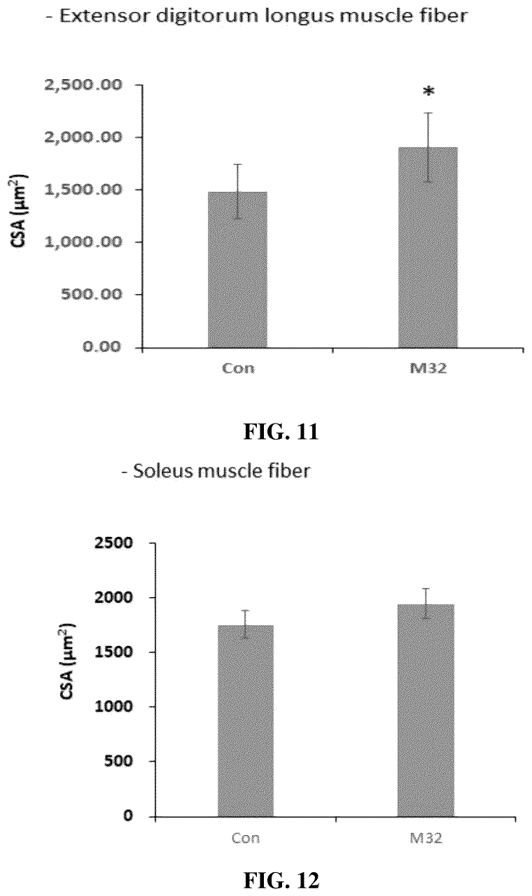

On the day of autopsy, a histopathological examination was performed by extracting calf muscle tissue and fixing it in a 10% neutral buffered formalin solution, and the fixed tissue was subjected to the process of trimming, dehydration, paraffin embedding, and sectioning to prepare a specimen for the histopathological examination. The specimen was stained by Hematoxylin & Eosin (H&E) staining, photographed using an optical microscope (Olympus BX53, Japan), and then quantified through an image analyzer. The results are shown in FIGS. 11 and 12. According to FIG. 11, it can be confirmed that the cross-sectional area of the muscle fiber size in the soleus was significantly increased in the G2 (L. paracasei_Myo-Act, 10 mg) experimental group compared to the lactic acid bacterium G1 (L. paracasei_Mcs) control group, which did not express an antigen, and according to FIG. 12, it can be confirmed that the size of the muscle fibers in the extensor digitorum longus was significantly increased in the G2 (L. paracasei_Myo-Act, 10 mg) experimental group compared to the G1 (L. paracasei_Mcs) control group.

### Example 3-2. Efficacy Evaluation in an Aged Mouse Model

### Example 3-2-1. Experimental Conditions

A nonclinical efficacy test was performed using an aged mouse model (C57BL/6J, 19 months old) that may exhibit the characteristics of sarcopenia. Specifically, killed Lactobacillus paracasei lactic acid bacteria were administered at a concentration of 10 mg/head, and after 15 weeks from the administration, sacrifice was performed to confirm the outcome variables. Serum Myostatin/Activin A IgG antibody was measured using ELISA, and a morphological analysis was performed within the muscle tissue by extracting the calf muscles. The composition of the test mice is shown in Table 5 below.

TABLE 5

| | | Number | | | amount of | volume of |
|---|---|---|---|---|---|---|
| | | of | | Route of | administration | administered solution |
| Group | Sex | animals | Test substance | administration | (mg/head) | (mL/head) |
| G1 | M | 10 | PBS | oral | 10 | 0.2 |
| | | | (Gavage Buffer) | administration | | |
| G2 | M | 10 | L. paracasei | oral | 10 | 0.2 |
| | | | (MCS) | administration | | |
| G3 | M | 10 | L. paracasei | oral | 10 | 0.2 |
| | | | (pAld-Myo-Act) | administration | | |

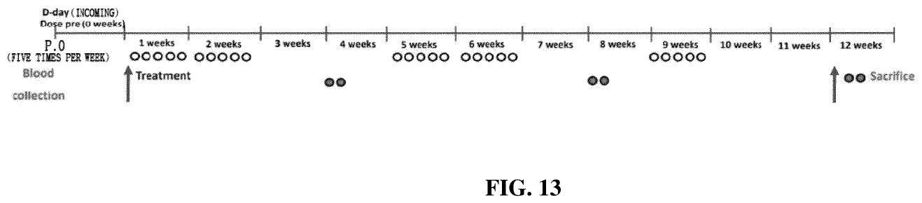

A solvent (PBS, G1) and lactic acid bacteria that did not express an antigen (L. paracasei_MCS, G2) were used as the control groups, and BLS-M32 (L. paracasei_Myo-Act, 10 mg, G3) was used as an experimental group. The test substance was administered a total of 25 times once per day for 5 days in Weeks 1, 2, 5, 6, and 9 from the day of starting the administration (FIG. 13).

A total of 25 doses were administered once per day for 5 days in Weeks 1, 2, 5, 6, and 9 from the start date (FIG. 13), and the experiment was performed by directly administering it into the stomach using an oral zonde in an amount of 200 μl/head.

### Example 3-2-2. Grip Strength Test and Rotarod Latency Test

In order to confirm the improvement of the grip strength and muscle endurance by the composition according to the present invention, a grip strength test and a rotarod latency test were performed.

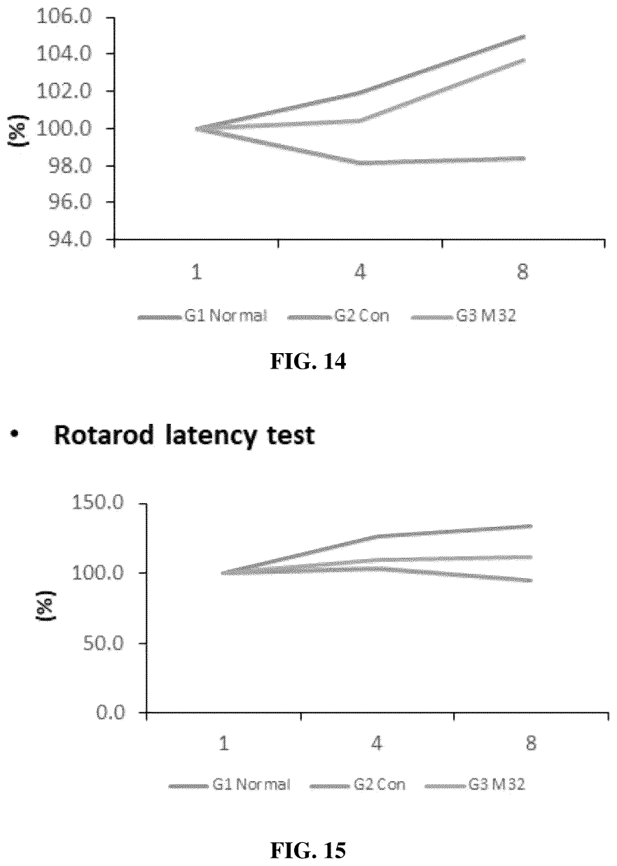

First, in the grip strength test to measure grip strength, the mouse was placed on a grip strength meter (47200, Ugo Basile, Italy) before and 1, 4, 8, 12, and 14 weeks after administering the test substance, and the tail was pulled to measure the grip strength. The results are shown in FIG. 14. It can be confirmed that the foreleg muscle strength (grip strength) was increased in the G3 (L. paracasei_Myo-Act, 10 mg) experimental group compared to the lactic acid bacterium G2 (L. paracasei_Mcs) control group, which did not express an antigen from four weeks after the administration of the test substance until the end of the administration.

Next, in the rotarod latency test to measure muscle endurance, the mice were placed on a rotarod-treadmill (JD-A-07MA5, B.S Technolab Inc., Korea) in Weeks 1, 4, 8, 12, and 14 after the administration of the test substance, and the measurement was performed for 300 sec while increasing the rotational speed at regular intervals from 4 rpm to 40 rpm. The results are shown in FIG. 15, and it can be confirmed that the muscular endurance increased in the G3 (L. paracasei_Myo-Act, 10 mg) experimental group compared to the lactic acid bacterium G2 (L. paracasei_Mcs) control group, which did not express an antigen from four weeks after the administration of the test substance until the end of the administration.

### Example 3-2-3. Histopathological Examination

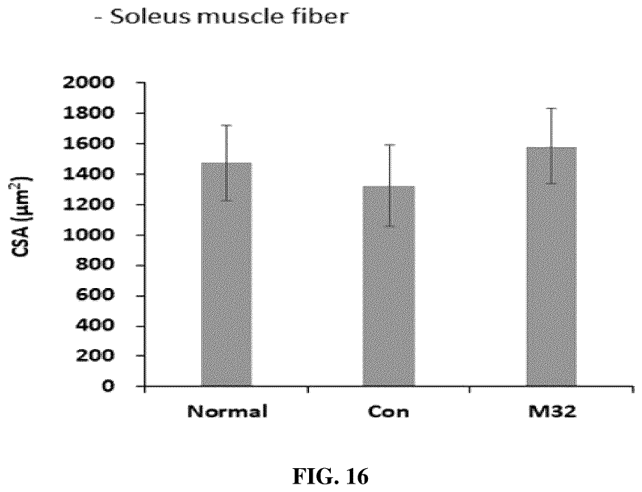

On the day of autopsy, a histopathological examination was performed by extracting calf muscle tissue and fixing it in a 10% neutral buffered formalin solution, and the fixed tissue was subjected to the process of trimming, dehydration, paraffin embedding, and sectioning to prepare a specimen for the histopathological examination. The specimen was stained by Hematoxylin & Eosin (H&E) staining, photographed using an optical microscope (Olympus BX53, Japan), and then quantified through an image analyzer. The results are shown in FIG. 16. According to FIG. 16, it can be confirmed that the cross-sectional area of the muscle fiber size in the soleus was significantly increased in the G3 (L. paracasei_Myo-Act, 10 mg) experimental group compared to the lactic acid bacterium G2 (L. paracasei_Mcs) control group, which did not express an antigen.

A microorganism cell surface display vector expressing myostatin and activin A as a bispecific antigen, and a microorganism transformed with the same according to the present invention have effects of alleviating, treating, and preventing sarcopenia.

As described above, the optimal embodiment is disclosed in the drawings and specifications. Although specific terms are used here, these are used only for the purpose of explaining the present invention and are not used to limit the meaning or scope of the present invention described in the claims. Therefore, those skilled in the art will understand that various modifications and other equivalent embodiments are possible therefrom. Therefore, the true scope of technical protection of the present invention should be determined by the technical spirit of the attached patent claims.

1. A pharmaceutical composition for alleviation, treatment, and prevention of sarcopenia, comprising, as an active ingredient, a microorganism transformed with a cell surface display vector operably linked with a gene encoding myostatin and activin A proteins.

2. The pharmaceutical composition for alleviation, treatment, and prevention of sarcopenia according to claim 1, wherein the cell surface display vector includes a pgsA gene.

3. The pharmaceutical composition for alleviation, treatment, and prevention of sarcopenia according to claim 1, wherein the myostatin has a gene sequence of SEQ ID NO: 1.

4. The pharmaceutical composition for alleviation, treatment, and prevention of sarcopenia according to claim 1, wherein the activin A has a gene sequence of SEQ ID NO: 3.

5. The pharmaceutical composition for alleviation, treatment, and prevention of sarcopenia according to claim 1, wherein the microorganism is a lactic acid bacterium.

6. The pharmaceutical composition for alleviation, treatment, and prevention of sarcopenia according to claim 5, wherein the lactic acid bacterium is Lactobacillus paracasei.
